(19) United States
(12) Patent Application Publication
(10) Pub. No.: US 2010/0040650 A1
(43) Pub. Date: Feb. 18, 2010

(54) Virus-Like paramyxovirus particles and vaccines

(76) Inventors: James E. Crowe, JR., Nashville, TN (US); Hoyin Mok, Redwood City, CA (US)

Correspondence Address: FULBRIGHT & JAWORSKI L.L.P., 600 CONGRESS AVE., SUITE 2400, AUSTIN, TX, 78701, US

(21) Appl. No.: 12/455,584

(22) Filed: Jun. 1, 2009

Related U.S. Application Data
(60) Provisional application No. 61/057,689, filed on May 30, 2008.

(51) Int. Cl. A61K 39/155; C07K 14/115; C12P 21/00; A61K 39/165

(52) U.S. Cl. 424/212.1; 530/350; 435/69.1; 424/211.1

(57) ABSTRACT

The present invention is directed to alphavirus virus-like particles produced by synthesizing in cell, including in vivo, structural proteins in the absence of other alphavirus proteins. In particular, these virus-like particules vaccines induce cellular and humoral immune responses that can block or inhibit alphavirus infections. Also disclosed are methods of vaccinating subjects with virus-like particles and vectors encoding the same.

This application claims benefit of priority to U.S. Provisional Application Ser. No. 61/057,689, filed May 30, 2008, the entire contents of which are hereby incorporated by reference.

This invention was made with government support under grant number R01 AI-59597 awarded by the National Institutes of Allergy and Infectious Disease and the National Institutes of Health. The government has certain rights in the invention.

### BACKGROUND OF THE INVENTION

1. Field of the Invention

The present invention relates generally to the fields of molecular biology, genetics and virology. More particularly, it concerns virus-like particles (VLPs) made of the structural proteins from a paramyxovirus. Vectors encoding the structural proteins are delivered to a cell, which express proteins that spontaneously form the VLPs, which generates an immune response. Vaccines and methods of protecting a subject from paramyxovirus infections also are provided.

2. Description of Related Art

Respiratory syncytial virus (RSV) is a paramyoxvirus that causes serious lower respiratory tract illness in infants and the elderly, making it a significant human pathogen. Significant morbidity and mortality for RSV is especially common in certain high-risk pediatric populations such as premature infants and infants with congenital heart or lung disorders. RSV bronchiolitis in infants is associated with recurrent wheezing and asthma later in childhood (Peebles, 2004; You et al., 2006). There are currently no FDA-approved vaccines for prevention of RSV disease by active immunization. Immunoprophylaxis by passive transfer of a humanized murine RSV fusion (F) protein-specific antibody is licensed for much of the high-risk infant population, but is not cost effective in otherwise healthy infants, who represent approximately 90% of those hospitalized with RSV.

Previous attempts to develop RSV vaccines have faced significant obstacles. An experimental formalin-inactivated RSV vaccine in the 1960s induced exacerbated disease and death in some vaccinated children during subsequent natural infection. It was shown subsequently that the formalin-inactivated RSV vaccine induced serum antibodies with poor neutralizing activity in infants (Murphy et al., 1986) and an atypical Th2-biased T cell response associated with enhanced histopathology following experimental immunization in small animals (Prince et al., 1986; Vaux-Peretz and Meignier, 1990). Treating RSV antigens with formaldehyde modifies the protein with carbonyl groups, which induce Th2-type responses preferentially and lead to enhanced disease (Moghaddam et al., 2006). Other attempts to generate RSV vaccines include using live-attenuated cold-adapted, temperature-sensitive mutant stains of RSV (Connors et al., 1995; Crowe et al., 1994a; Crowe et al., 1996a; Crowe et al., 1994b; Crowe et al., 1995; Crowe et al., 1993; Crowe et al., 1996b; Crowe et al., 1998; Firestone et al., 1996; Hsu et al., 1995; Juhasz et al., 1997; Karron et al., 1997; Karron et al., 2005), protein subunit vaccines coupled with adjuvant (Power et al., 1997; Welliver et al., 1994; Walsh, 1993; Homa et al., 1993) and RSV proteins expressed from recombinant viral vectors including vaccinia virus (Olmsted et al., 1986; Wyatt et al., 1999), adenovirus (Hsu et al., 1992), vesicular stomatitis virus (Kahn et al., 2001), Semliki Forest virus (Chen et al., 2002), bovine/human parainfluenza type 3 (Haller et al., 2003), Sendai virus (Takimoto et al., 2004) and Newcastle disease virus (Martinez-Sobrido et al., 2006).

The two surface glycoproteins of RSV, fusion (F) protein and attachment (G) protein, are the major antigenic targets for neutralizing antibodies. Neutralizing antibodies are sufficient to protect the lower respiratory tract (Connors et al., 1991). F and G proteins, therefore, have been used separately or in combination in many experimental RSV vaccines. Immunization with purified F protein alone or F protein expressed from a recombinant viral vector such as vaccinia virus induces RSV-specific neutralizing antibodies, CD8+ cytotoxic T lymphocytes and protection against subsequent RSV challenge in mice or cotton rats (Olmsted et al., 1986). Vaccination with G protein alone, however, often induces only partial protection against RSV challenge. In mice, the immune response against G is associated with eosinophilia and the induction of TH2 type CD4+ lymphocytes in some experiments (Tebbey et al., 1998; Johnson et al., 1998; Hancock et al., 1996).

Human metapneumovirus (hMPV) is a paramyxovirus recently discovered in young children with respiratory tract disease (van den Hoogen et al., 2001). Subsequent studies show that hMPV is a causative agent for both upper and lower respiratory tracts infections in infants and young children (Boivin et al., 2002; Esper et al., 2004; Esper et al., 2004; Falsey et al., 2003; Williams et al., 2005; Williams et al., 2004). The spectrum of clinical illness ranges from cough and wheezing to bronchiolitis and pneumonia, similar to those seen in respiratory syncytial virus (RSV) and parainfluenza virus (PIV) infections. Children and adults with comorbid conditions, such as those with congenital heart and lung diseases, cancer and immunodeficiency, are particular at risk for acute respiratory disease from hMPV infection (Pelletier et al., 2002; Williams et al., 2005). Epidemiology studies, although not completely defined, has put hMPV infection incidence rate at 5-15% in young children (Boivin et al., 2002; Falsey et al., 2003; Williams and Harris, 2004; Pelletier et al., 2002; McAdam et al., 2004; Osterhaus and Fouchier, 2003). Recurrent infection of hMPV has also been documented (Ebihara et al., 2004). This, in combination with RSV and PIV, represents the leading causes for acute viral respiratory tract infections in this population and warrants the development of vaccine against this recently discovered virus.

Similarly to RSV, fusion F and attachment G proteins are the major surface glycoproteins on hMPV. Genetic analysis put hMPV into two subgroups (A and B) based on sequence comparison of these two genes in various clinical isolates (Bastien et al., 2003; Biacchesi et al., 2003). The subgroups are further divided into sublineages A1, A2, B1 and B2. The percent amino acid homology in the F protein reaches >95% and is highly conserved between the subgroups (Boivin et al., 2004; Skiadopoulos et al., 2004). G protein, however, shows significant amino acid diversification with homology ranging from 34-100% depending on inter- or intra- subgroup comparisons (Biacchesi et al., 2003; Bastien et al., 2004). In RSV, F and G proteins are the major antigenic targets for neutralizing antibodies. High titers of serum neutralizing antibodies are sufficient to protect the lower respiratory tract for RSV infection (Connors et al., 1991). Therefore, F and G proteins had been used singly or in combinations in various experimental vaccines.

As with RSV, a number of vaccines have been developed for hMPV. These include subunit F vaccine (Cseke et al., 2007), live-attenuated hMPV with gene deletions (Biacchesi et al., 2004) and a chimeric, live-attenuated PIV vaccine that incorporates the hMPV F, G or SH gene (Skiadopoulos et al., 2006; Tang et al., 2005; Tang et al., 2003). Although proven to be immunogenic in animal models, there are significant hurdles for some of these vaccines to be used in very young infants, which is one of the principle targets of hMPV vaccines. The presence of circulating maternal antibodies against most of the candidate vaccines and viral vectors is of concern and may blunt the efficacies of these vaccines in vivo. Furthermore, the ability to generate a mucosal response is pertinent to successful immunization against respiratory viruses.

Thus, a key determinant for optimal vaccination against respiratory viruses, such as RSV and human metapneumovirus (hMPV), is the ability of the vaccine to generate mucosal immunity. This goal can be achieved by using a topical route for vaccination or possibly by use of a vaccine construct that preferentially induces mucosal responses. Protection in the upper respiratory tract usually results only from immunization by the intranasal route, which can result in the induction of virus-specific mucosal IgA antibodies. However, as of yet a successful vaccine against paramyxoviruses like RSV, HPIV and hMPV has yet to be achieved.

### SUMMARY OF THE INVENTION

Thus, in accordance with the present invention, there is provided a paramyxovirus virus-like particle comprising paramyxovirus matrix protein (M), nucleoprotein (N), phosphoprotein (P) and fusion protein (F), but excluding all other paramyxovirus proteins. The paramyxovirus virus-like particle may be respiratory syncytial virus, metapneumovirus, parainfluenza virus, or measles virus.

In another embodiment, there is provided a method of producing a paramyxovirus virus-like particle comprising (a) providing one or more expression constructs encoding paramyxovirus matrix protein (M), nucleoprotein (N), phosphoprotein (P) and fusion protein (F); (b) transferring the one or more expression constructs into a host cell; and (c) culturing the host cell under conditions supporting expression of M, N, P and F proteins. Each of the M, N, P and F may be encoded on one distinct expression constructs, two distinct expression constructs or three distinct expression constructs. For example three of M, N, P and F may be coded on one construct, while the fourth is coded on a second; alternatively, two may be coded on a single construct and two others may be coded on a second construct, or a second and third construct, respectively. Still, further, each of the M, N, P and F may be encoded on distinct (four) expression constructs. The one or more expression constructs is a viral expression construct, such as an alphavirus construct. The host cell may be a human cell. Transferring nay comprise DNA gene gun transfer. The one or more expression constructs are comprised in a lipid formulation. The paramyxovirus may be a respiratory syncytial virus, metapneumovirus, parainfluenza virus, or measles virus. The paraymyxovirus may more generally be from the subfamily Pneumovirinae, from the genus Pneumovirus, or from the genus Metapneumovirus.

In still yet another embodiment, there is provided a method of inducing a paramyxovirus immune response in a subject comprising (a) providing one or more expression constructs encoding paramyxovirus matrix protein (M), nucleoprotein (N), phosphoprotein (P) and fusion protein (F); (b) administering the one or more expression constructs to the subject. Each of the M, N, P and F may be encoded on one distinct expression constructs, two distinct expression constructs or three distinct expression constructs. For example three of M, N, P and F may be coded on one construct, while the fourth is coded on a second; alternatively, two may be coded on a single construct and two others may be coded on a second construct, or a second and third construct, respectively. Still, further, each of the M, N, P and F may be encoded on distinct (four) expression constructs. The one or more expression constructs is a viral expression construct, such as an alphavirus construct. The subject may be a human subject, such as an infant or an immunocompromised subject. Transferring may comprise DNA gene gun transfer. The one or more expression constructs may be comprised in a lipid formulation. Administering may comprise intramuscular or intradermal injection. The paramyxovirus may be respiratory syncytial virus, metapneumovirus, parainfluenza virus, or measles virus. The paraymyxovirus may more generally be from the subfamily Pneumovirinae, from the genus Pneumovirus, or from the genus Metapneumovirus. The method may further comprise assessing an immune response to M, N, P and/or F proteins in the subject.

In still yet an additional embodiment, there is provided a method of immunizing a subject comprising administering to the subject a paramyxovirus virus-like particle comprising matrix protein (M), nucleoprotein (N), phosphoprotein (P) and fusion protein (F), but excluding all other paramyxovirus proteins. The paramyxovirus may be respiratory syncytial virus, metapneumovirus, parainfluenza virus, or measles virus. The paraymyxovirus may more generally be from the subfamily Pneumovirinae, from the genus Pneumovirus, or from the genus Metapneumovirus. Administering may comprise intramuscular or intradermal injection. The subject may be a human subject, such as an infant or an immunocompromised subject. The method may further comprise administering to the subject an adjuvant. The method may also further comprise assessing an immune response to M, N, P and/or F proteins in the subject.

Administration may also comprise intranasal inhalation or subcutaneous injection of the vaccine. The method may further comprise administering the vaccine a second time or a third time. Assessing an immune response to the VLP may be by RIA, ELISA, immunohistochemistry or Western blot. The immune response in the animal may be a humoral response, such as a mucosal IgA response, or a serum IgG response. The serum IgG response may be neutralizing. The immune response may be cellular, such as a balanced Th1/Th2 response.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Confocal microscopy of RSV proteins expressed in epithelial cells in culture. Transfection with 4 plasmids (encoding F, M, N, and P) leads to dramatic formation of viral filament.

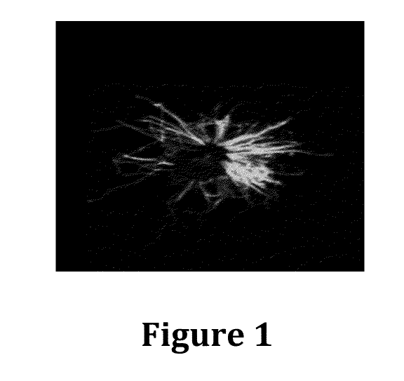

FIG. 2—Inducible gene expression system. Th inducible gene expression system (pTRE) was used to create a virus-like particle expressio cell line.

FIG. 3—Production and purification of RSV virus-like particles.

FIG. 4—Detection of RSV structural proteins in lodixanol fractions by Western blotting. After membrane flotation, 200 μl fractions were collected. Proteins in the fractions were separated by SDS-PAGE and transferred onto a PVDF membrane. Viral proteins were detected by RSV-specific antibodies using standard Western blot protocol.

FIG. 5—Supernatant from transfected ³⁵S labeled 293T cells contains RSV virus-like particles. 293T cells were labeled with ³⁵S-methionine and cysteine after transfection. Supernatant was harvested 24 hours later and purified with a discontinuous gradient. Fractions were collected and immunoprecipitated with RSV-F antibodies. Immuno precipitated proteins were separated with SDS-PAGE and transferred onto PVDF membranes. Autoradiography was used to detected ³⁵S-labeled proteins.

FIG. 6—Vaccination of BALB/c ice with 500 ng RSV VLPs induces elevated serum levels of RSV-F specific antibodies as detected by ELISA.

FIGS. 7A-B—VRP induced robust RSV F-specific and neutralizing antibody responses in mice with pre-existing neutralizing RSV antibodies. Naïve mice (filled symbols) or mice passively transferred with neutralizing RSV antibodies (unfilled symbols) were inoculated once with 10⁶ plaque forming units of RSV intranasally or 10⁶ infectious units of VRP-RSV.F intranasally or intramuscularly. (FIG. 7A) Sera from vaccinated mice were obtained 28 days post-vaccination. RSV-F specific enzyme-linked immunosorbent assay (ELISA) was performed on serially diluted sera with HRP-conjugated anti-mouse Ig antibodies. Amount of binding was determined from absorbance of HRP-substrate at λ=450 nm. Murine antibody ELISA titers were expressed as the reciprocal titer of serum in which the absorbance was twice the background signal. (FIG. 7B) Sera from vaccinated mice also were tested for RSV neutralizing activity in a plaque reduction assay. Serum neutralizing activity is expressed as the geometric mean titer (GMT) for 60% reduction of RSV plaques in HEp-2 cell monolayer cultures. Lines denote the geometric mean titer within a group.

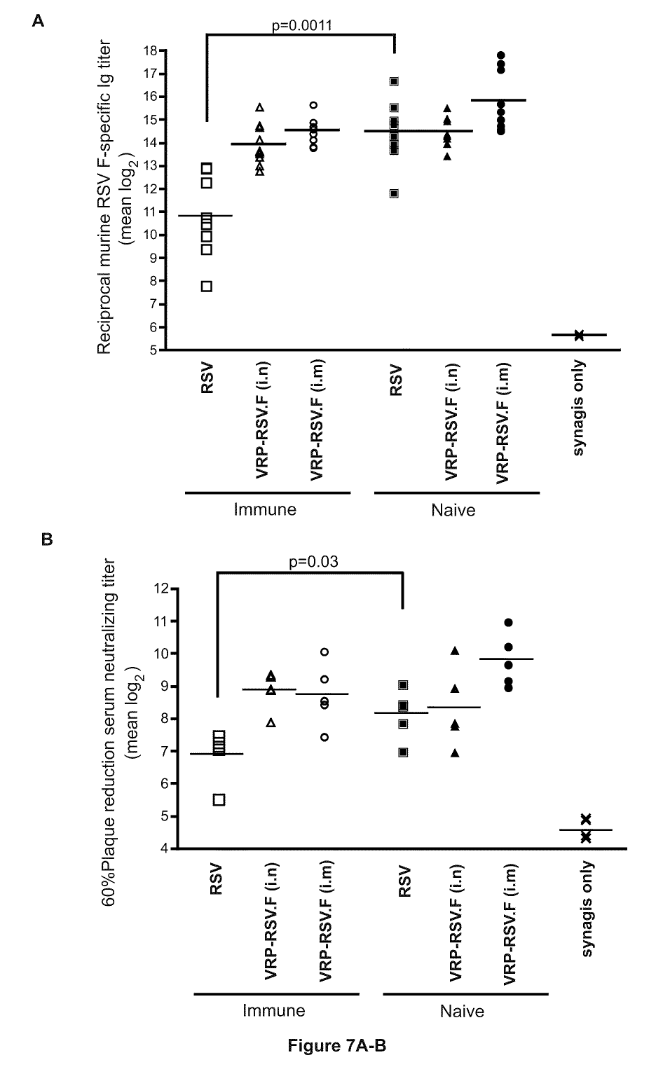

FIG. 8—VRP elicited a similar frequency of RSV F specific B cells in the lungs and mediastinal lymph nodes in immune versus naïve mice. BALB/c mice with (o) or without (x) pre-existing neutralizing RSV antibodies were inoculated once with VRP or wild-type RSV and then challenged with live RSV on day 28. Lymphocytes were harvested 3 days after challenge (day 31) from the lungs and mediastinal lymph nodes. Numbers of RSV F-specific Ig-secreting B cells were evaluated using a B cell ELISPOT. Spots were counted with an automated counting device and are expressed as numbers of spot forming cells per 10⁶ cells. Each experimental group contained 5 animals. Each horizontal line denotes the geometric mean of the group.

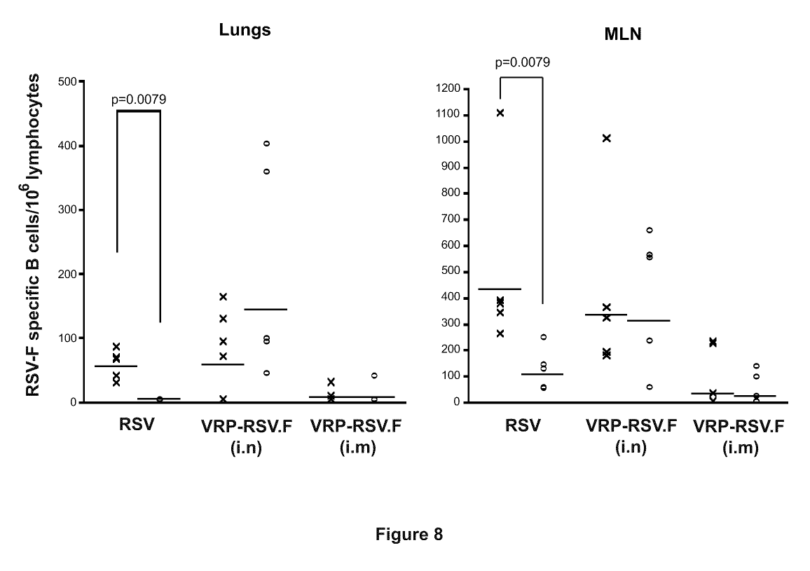

FIG. 9—Similar levels of RSV F-specific lymphocytes and splenocytes were induced after RSV challenge in the lungs, mediastinal lymph nodes and spleens of naïve versus immune mice immunized with VRP. BALB/c mice with (o) or without (x) pre-existing neutralizing RSV antibodies were inoculated once with VRP or wild-type RSV and challenged with live RSV 28 days later. Lymphocytes and splenocytes were harvested from the lungs, mediastinal lymph nodes or spleens 7 days after vaccination or 3 days after RSV challenge (i.e., day 31). A suspension of 1×10⁵ cells was stimulated with RSV F (aa. 85-93) peptide in vitro for 20 hours and the numbers of IFN-γ spot forming cells were quantified by an IFN-γ ELISPOT assay. Spots were counted with an automated counting device and are expressed as numbers of spot forming cells per 10⁶ cells. Each experimental group contained 5 animals. Each horizontal line denotes the geometric mean of the group.

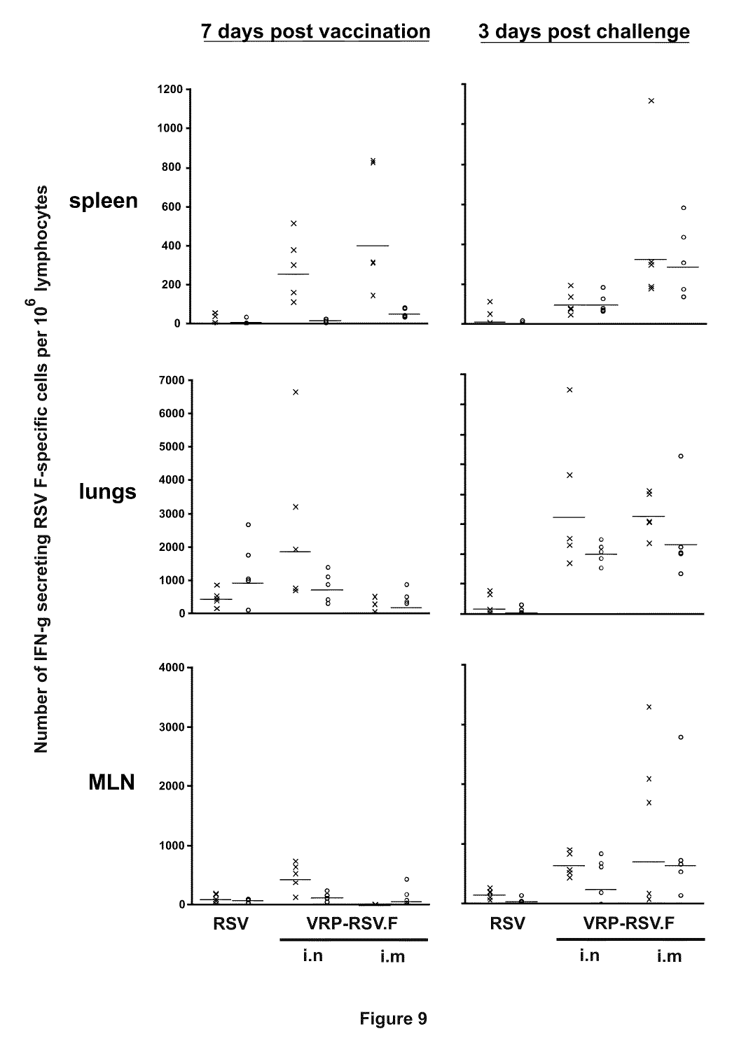

### DESCRIPTION OF ILLUSTRATIVE EMBODIMENTS

The present invention proposes the simultaneous inoculation of multiple structural virus genes into a mammalian host in order to express in vivo each of the multiple virus proteins, which comprise the protective antigens and the minimal requirements of particle formation, thereby leading to in vivo formation of virus-like particles (VLPs) and filaments, facilitating immunization of the mammalian host. While immunization with proteins, virus-like particles, and gene delivery are known, the present invention centers on the in vivo formation of VLPs and filaments for the purpose of vaccination.

Recent efforts have shown that presentation of virus proteins in the context of a particle that looks like a virion particle, but lacks virus genome (noninfectious “virus-like particles”), may better present protective antigens to the immune system. Such presentations achieve the authentic conformation of multimeric protein complexes in a way that purified proteins can not achieve. Producing and purifying VLPs in large scale using existing technology is expensive and challenging, and may alter the conformation of antigens. Transport and storage of thermolabile purified protein constructs in the developing world can be challenging. The essence of this invention is to immunize with multiple virus genes leading to the formation of VLPs in vivo, thus providing effective immunization.

The first iteration that proposed is a vaccine for human respiratory syncytial virus (hRSV). hRSV is the most important cause of severe lower respiratory tract infection (LRI) in infants and young children throughout the world, and also is a major cause of morbidity in the elderly and immunocompromised. There is currently no licensed vaccine for this major human pathogen. The inventors have generated RSV VLPs by transfecting plasmids encoding four of the ten RSV genes—matrix protein (M), nucleoprotein (N), phosphoprotein (P) and fusion protein (F)—into cells in vitro. The same approach will be used to generate VLPs in vivo by injecting animals with genes encoding the same four RSV proteins that have proved sufficient to form VLPs in vitro.

A VLP DNA-based vaccine has major safety advantages over current live vaccine strategies since it does not replicate. VLPs could be commercialized as a non-replicating protein vaccine for use in humans to prevent RSV. Formulated as DNA vaccines, this strategy could yield a highly thermostable vaccine preparation that is inexpensive to manufacture. The use of this approach would not be limited to RSV, however, and may be extended to paramyxoviruses generally given their structural and functional homology. The approach is to identify the minimal protein requirements needed for formation of VLPs containing protective antigens for any given virus, then to immunize with genes encoding those proteins in order to form VLPs in vivo for the purposes of vaccination. The method of gene delivery could be DNA immunization, virus vectors, or other strategies, including delivery of the VLPs themselves. These and other aspects of the invention are addressed below.

### I. PARAMYXOVIRUSES

Paramyxoviruses are viruses of the Paramyxoviridae family of the Mononegavirales order; they are negative-sense single-stranded RNA viruses responsible for a number of human and animal diseases. Virions are enveloped and can be spherical, filamentous or pleomorphic. Fusion proteins and attachment proteins appear as spikes on the virion surface. Matrix proteins inside the envelope stabilise virus structure. The nucleocapsid core is composed of the genomic RNA, nucleocapsid proteins, phosphoproteins and polymerase proteins.

The genome consists of a single segment of negative-sense RNA, 15-19 kilobases in length and containing 6-10 genes. Extracistronic (non-coding) regions include: a 3′ leader sequence, 50 nucleotides in length which acts as a transcriptional promoter; and a 5′ trailer sequence, 50-161 nucleotides long. Intergenomic regions between each gene which are three nucleotides long for morbillivirus, respirovirus and henipavirus, variable length (1-56 nucleotides) for rubulavirus and pneumovirinae. Each gene contains transcription start/stop signals at the beginning and end which are transcribed as part of the gene. Gene sequences within the genome are conserved across the family due to a phenomenon known as transcriptional polarity (see Mononegavirales) in which genes closest to the 3′ end of the genome are transcribed in greater abundance than those towards the 5′ end. This mechanism acts as a form of transcriptional regulation. The gene sequence is: Nucleocapsid-Phosphoprotein-Matrix-Fusion-Attachment-Large (polymerase).

The nucleocapsid protein associates with genomic RNA (one molecule per hexamer) and protects the RNA from nuclease digestion. The phosphoprotein binds to the N and L proteins and forms part of the RNA polymerase complex. The matrix protein assembles between the envelope and the nucleocapsid core, it organises and maintains virion structure. The fusion protein projects from the envelope surface as a trimer, and mediates cell entry by inducing fusion between the viral envelope and the cell membrane by class I fusion. One of the defining characteristics of members of the paramyxoviridae family is the requirement for a neutral pH for fusogenic activity.

The cell attachment proteins (H/HN/G) span the viral envelope and project from the surface as spikes. Many have been shown to bind to sialic acid on the cell surface and facilitate cell entry. Proteins are designated H for morbilliviruses and henipaviruses as they possess haemagglutination activity, observed as an ability to cause red blood cells to clump. UN attachment proteins occur in respiroviruses and rubulaviruses. These possess both haemagglutination and neuraminidase activity which cleaves sialic acid on the cell surface, preventing viral particles from reattaching to previously infected cells. Attachment proteins with neither haemagglutination nor neuraminidase activity are designated G (glycoprotein). These occur in members of pneumovirinae. The large protein is the catalytic subunit of RNA dependent RNA polymerase (RDRP).

The subfamily Pneumovirinae contains two important human pathogens, respiratory syncytial virus from the genus Pneumovirus, and metapneumovirus from the genus Metapneumovirus. Virions have an envelope and a nucleocapsidand are spherical to pleomorphic; however, filamentous and other forms are common. The virions are about 60-300 nm in diameter and 1000-10000 nm in length. The Mr of the genome constitutes 0.5% of the virion by weight. The genome is not segmented and contains a single molecule of linear negative-sense, single-stranded RNA. Virions may also contain occasionally a positive sense single-stranded copy of the genome. The complete genome is about 15,300 nucleotides long.

Other paramyxoviruses include Newcastle disease virus, Hendravirus; Nipahvirus, Measles virus; Rinderpest virus, Canine distemper virus, phocine distemper virus, Sendai virus, Human parainfluenza viruses 1-4, Mumps virus; Simian parainfluenza virus 5, Menangle virus, Tioman virus, Tupaia paramyxovirus, Bovine respiratory syncytial virus, Avian pneumovirus, Fer-de-Lance virus, Nariva virus, Salem virus, J virus, Mossman virus and Beilong virus.

A. RSV

Human respiratory syncytial virus (hRSV) is a negative-sense, single-stranded RNA virus that causes respiratory tract infections in patients of all ages. It is the major cause of lower respiratory tract infection during infancy and childhood. In temperate climates there is an annual epidemic during the winter months. In tropical climates, infection is most common during the rainy season. In the United States, 60% of infants are infected during their first RSV season, and nearly all children will have been infected with the virus by 2-3 years of age. Natural infection with RSV does not induce protective immunity, and thus people can be infected multiple times. Sometimes an infant can become symptomatically infected more than once even within a single RSV season. More recently, severe RSV infections have increasingly been found among elderly patients as well.

For most people, RSV produces only mild symptoms, often indistinguishable from common colds and minor illnesses. The Centers for Disease Control consider RSV to be the “most common cause of bronchiolitis and pneumonia among infants and children under 1 year of age.” For some children, RSV can cause bronchiolitis, leading to severe respiratory illness requiring hospitalization and, rarely, causing death. This is more likely to occur in patients that are immunocompromised or infants born prematurely. Other RSV symptoms common among infants include listlessness, poor or diminished appetite, and a possible fever.

Recurrent wheezing and asthma are more common among individuals who suffered severe RSV infection during the first few months of life than among controls; whether RSV infection sets up a process that leads to recurrent wheezing or whether those already predisposed to asthma are more likely to become severely ill with RSV is a matter of considerable debate.

As the virus is ubiquitous in all parts of the world, avoidance of infection is not possible. Epidemiologically, a vaccine would be the best answer. Unfortunately, vaccine development has been fraught with spectacular failure and with difficult obstacles. Researchers are working on a live, attenuated vaccine, but at present no vaccine exists. However, palivizumab (brand name Synagis), a moderately effective prophylactic drug is available for infants at high risk. Palivizumab is a monoclonal antibody directed against RSV proteins. It is given by monthly injections, which are begun just prior to the RSV season and are usually continued for five months. RSV prophylaxis is indicated for infants that are premature or have either cardiac or lung disease.

Ribavirin, a broad-spectrum antiviral agent, was once employed as adjunctive therapy for the sickest patients; however, its efficacy has been called into question by multiple studies, and most institutions no longer use it. Treatment is otherwise supportive care only with fluids and oxygen until the illness runs its course. Amino acid sequences 200-225 and 255-278 of the F protein of human respiratory syncytial virus (HRSV) are T cell epitopes (Corvaisier et al., 1993). Peptides corresponding to these two regions were synthesized and coupled with keyhole limpet haemocyanin (KLH). The two conjugated proteins were administered intranasally to BALB/c mice alone or together with cholera toxin B (CTB). ELISAs revealed that the mixture of the conjugates with CTB increased not only the systemic response but also the mucosal immune response of the saliva. The systemic response was lower and the mucosal immune response was undetectable in mice immunized with the conjugates on their own. These results suggest that these two peptide sequences are effective epitopes for inducing systemic and mucosal immune responses in conjunction with CTB, and may provide the basis for a nasal peptide vaccine against RSV for human use.

B. MPV

Human metapneumovirus (hMPV) was isolated for the first time in 2001 in the Netherlands by using the RAP-PCR technique for identification of unknown viruses growing in cultured cells. hMPV is a negative single-stranded RNA virus of the family Paramyxoviridae and is closely related to the avian metapneumovirus (AMPV) subgroup C. It may be the second most common cause (after the RSV) of lower respiratory infection in young children.

Compared with RSV, infection with human metapneumovirus tends to occur in slightly older children and to produce disease that is less severe. Co-infection with both viruses can occur, and is generally associated with worse disease. Human metapneumovirus accounts for approximately 10% of respiratory tract infections that are not related to previously known etiologic agents. The virus seems to be distributed worldwide and to have a seasonal distribution with its incidence comparable to that for the influenza viruses during winter. Serologic studies have shown that by the age of five, virtually all children have been exposed to the virus and reinfections appear to be common. Human metapneumovirus may cause mild respiratory tract infection however small children, elderly and immunocompromised individuals are at risk of severe disease and hospitalization. The genomic organisation of hMPV is analogous to RSV, however hMPV lacks the non-structural genes NS1 and NS2 and the hMPV antisense RNA genome contains eight open reading frames in slightly different gene order than RSV (viz. 3′-N-P-M-F-M2-SH-G-L-5′). hMPV is genetically similar to the avian pneumoviruses A, B and in particular type C. Phylogenetic analysis of hMPV has demonstrated the existence of two main genetic lineages termed subtype A and B containing within them the subgroups A1/A2 and B1/B2 respectively. The identification of hMPV has predominantly relied on reverse-transcriptase polymerase chain reaction (RT-PCR) technology to amplify directly from RNA extracted from respiratory specimens. Alternative more cost effective approaches to the detection of hMPV by nucleic acid-based approaches have been employed and these include: 1) detection of hMPV antigens in nasopharyngeal secretions by immunofluorescent-antibody test 2) the use of immunofluorescence staining with monoclonal antibodies to detect hMPV in nasopharyngeal secretions and shell vial cultures 3) immunofluorescence assays for detection of hMPV-specific antibodies 4) the use of polyclonal antibodies and direct isolation in cultures cells.

C. Parainfluenza Virus

Human parainfluenza viruses (HPIVs) are a group of four distinct serotypes of single-stranded RNA viruses belonging to the paramyxovirus family. They are the second most common cause of lower respiratory tract infection in younger children. Repeated infection throughout the life of the host is not uncommon. Symptoms of later breakouts include upper respiratory tract illness as in a cold and sore throat. The incubation period of all four serotypes is 1 to 7 days. Parainfluenza viruses can be detected via cell culture, immunofluorescent microscopy, and PCR.

The HPIVs are negative-sense, single-stranded RNA viruses that possess fusion and hemagglutinin-neuraminidase glycoprotein “spikes” on their surface. The virion varies in size (average diameter between 150 and 300 nm) and shape, is unstable in the environment (surviving a few hours on environmental surfaces), and is readily inactivated with soap and water, hence, there is great value in handwashing for combating spread of HPIVs.

Though no vaccines currently exist, research into vaccines for HPIV-1, -2, and -3 is underway. Parainfluenza viruses last only a few hours in the environment and are inactivated by soap and water. The four serotypes include: HPIV-1 (most common cause of croup; also other upper and lower respiratory tract illnesses typical); HPIV-2 (causes croup and other upper and lower respiratory tract illnesses); HPIV-3 (associated with bronchiolitis and pneumonia); and HPIV-4 (includes subtypes 4a and 4b).

Like RSV, human parainfluenza viruses can cause repeated infections throughout life. These infections are usually manifested by an upper respiratory tract illness (such as a cold or sore throat). They can also cause serious lower respiratory tract disease with repeat infection (including pneumonia, bronchitis, and bronchiolitis), especially among the elderly, and among patients with compromised immune systems. HPIVs are ubiquitous and infect most people during childhood. The highest rates of serious HPIV illnesses occur among young children. Serologic surveys have shown that 90% to 100% of children aged 5 years and older have antibodies to HPIV-3, and about 75% have antibodies to HPIV-1 and HPIV-2. The different HPIV serotypes differ in their clinical features and seasonality. HPIV-1 causes biennial outbreaks of croup in the fall (presently in the United States during odd numbered years). HPIV-2 causes annual or biennial fall outbreaks. HPIV-3 peak activity occurs during the spring and early summer months each year, but the virus can be isolated throughout the year.

The diagnosis of HPIV can be confirmed in two ways: (1) by isolation and identification of the virus in cell culture or direct detection of the virus in respiratory secretions using immunofluorescence, enzyme immunoassay, or polymerase chain reaction (PCR) assay, and (2) by demonstration of a significant rise in specific IgG antibodies between appropriately collected paired serum specimens or specific IgM antibodies in a single serum specimen.

D. Measles Virus

Measles, also known as rubeola, is a disease caused by a virus, specifically a paramyxovirus of the genus Morbillivirus. Measles is spread through respiration (contact with fluids from an infected person's nose and mouth, either directly or through aerosol transmission), and is highly contagious—90% of people without immunity sharing a house with an infected person will catch it. Airborne precautions should be taken for all suspected cases of measles. The incubation period usually lasts for 4-12 days (during which there are no symptoms). Infected people remain contagious from the appearance of the first symptoms until 3-5 days after the rash appears.

Reports of measles go as far back to at least 600 B.C. In roughly the last 150 years, measles has killed an estimated 200 million people worldwide. In 1954, the virus causing the disease was isolated from an 11-year old boy from the United States, and adapted and propagated on chick embryo tissue culture. To date, 21 strains of the measles virus have been identified. Licensed vaccines to prevent the disease became available in 1963.

The measles is a highly contagious airborne pathogen which spreads primarily via the respiratory system. The virus is transmitted in respiratory secretions, and can be passed from person to person via aerosol droplets containing virus particles, such as those produced by a coughing patient. Once transmission occurs, the virus infects the epithelial cells of its new host, and may also replicate in the urinary tract, lymphatic system, conjunctivae, blood vessels, and central nervous system. Patients with the measles should be placed on droplet precautions. Humans are the only known natural hosts of measles, although the virus can infect some non-human primate species.

### II. EXPRESSION VECTORS AND DELIVERY SYSTEMS

Throughout this application, the term “expression construct” is meant to include any type of genetic construct containing a nucleic acid coding for a gene product in which part or all of the nucleic acid encoding sequence is capable of being transcribed. In such embodiments, the nucleic acid encoding the gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

Another element that may be included in an expression construct is a termination signal comprised of the DNA sequences involved in specific termination of an RNA transcript by an RNA polymerase. A terminator may be necessary in vivo to achieve desirable message levels. In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

As mentioned above, particularly eukaryotic expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). According to the present invention, two nucleic acids segments encoding the two portions of Cre may be incorporated into the same expression construct, thereby permitting expression of both with a single construct and a single selectable marker.

IRES elements from two members of the picanovirus family (polio and encephalomyocarditis) have been described (Pelletier and Sonenberg, 1988), as well an IRES from a mammalian message (Macejak and Samow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

A. VEE Vaccine Delivery System The present invention may utilize an alphavirus delivery system based on virus replicon particles (VRPs) of Venezuelan equine encephalitis (VEE) virus, an RNA virus of the Togaviradae family VRPs are non-replicating particles developed by Pushko et al. in 1997, which been used successfully and safely in immunization and challenge studies for a wide range of viral and bacterial pathogens in animal model systems (Pushko et al., 1997; Balasuriya et al., 2002; Burkhard et al., 2002; Gipson et al., 2003; Harrington et al., 2002; Hevey et al., 1998; Johnston et al., 2005; Lee et al., 2002; Pushko et al., 2001; Schultz-Chemy et al., 2000; Velders et al., 2001; Wang et al., 2005), including influenza virus, Lassa fever virus, Marburg virus, and most recently HIV. Importantly, these particles have been shown to induce mucosal immune responses after parenteral or intradermal inoculation in animals (Harrington et al., 2002; Davis et al., 1996). Currently this vector system is being tested in phase I clinical trials in humans to determine the safety of candidate vaccine encoding HIV antigens (Davis et al., 2002; Williamson et al., 2003).

VRPs are intact, replication-deficient VEE virus particles that contain a modified positive-sense RNA viral genome designed to express only the heterologous antigens. These particles are produced in a cellular packaging system in which structural proteins are supplied in trans and only the modified viral genome is packaged into an intact VRP. The resulting replicons express high levels of antigens in infected cells and induce humoral and cellular immune responses in vivo (Pushko et al., 1997). VRPs possess the ability to target dendritic cells and induce mucosal responses (MacDonald and Johnston, 2000), which is optimal for protecting against viruses at the respiratory tract mucosa. Although the mechanism underlying this unique mucosal immunogenicity of VRPs is not completely understood, significant numbers of cells secreting antigen-specific IgA have been detected in the mucosa in immunized animals following VRP immunization (Pushko et al., 1997; Harrington et al., 2002; Johnston et al., 2005; Davis et al., 1996; Davis et al., 2002). Moreover, when VRP particles were co-administered with microbial antigens, they exhibit adjuvant activity in the systemic and mucosal immune compartments (Thompson et al., 2006).

The present inventors have previously generated VEE replicon vaccine vectors for single antigens for both RSV and hMPV and tested them to determine whether effective mucosal protection could be induced against these pathogens following intranasal immunization. VRPs encoding the RSV F protein induced both systemic and mucosal antibody responses. These VRPs also induced antigen-specific T cells in both the lungs and spleens of immunized animals. The T cell responses were Th1/Th2 balanced, and aggravated histopathology was not observed. In addition, these animals were protected completely following challenge with wild-type RSV. In contrast, animals vaccinated with VRPs encoding the RSV attachment protein G were only partially protected. These findings provide proof-of-principle that VEE VRPs expressing the RSV F protein can be used to prevent RSV infection.

Additional details of this vector system and its use can be found in U.S. Patent Publication 2002/014975 A1 (incorporated by reference), as well as on the World Wide Web at alphavax.com. Other patent documents that are relied upon to provide a description of this system include U.S. Pat. Nos. 5,185,440, 5,505,947, 5,643,576, 5,792,462, 6,156,558, 6,521,235, 6,531,135, 6,541,010, 6,738,939, 7,045,335 and 7,078,218, each of which are incorporated herein by reference.

The following discussion is derived from U.S. Pat. No. 7,045,335:
. . . The terms “alphavirus replicon particles,” “virus replicon particles” or “recombinant alphavirus particles,” used interchangeably herein, mean a virion-like structural complex incorporating an alphavirus replicon RNA that expresses one or more heterologous RNA sequences. Typically, the virion-like structural complex includes one or more alphavirus structural proteins embedded in a lipid envelope enclosing a nucleocapsid that in turn encloses the RNA. The lipid envelope is typically derived from the plasma membrane of the cell in which the particles are produced. Preferably, the alphavirus replicon RNA is surrounded by a nucleocapsid structure comprised of the alphavirus capsid protein, and the alphavirus glycoproteins are embedded in the cell-derived lipid envelope. The alphavirus replicon particles are infectious but replication-defective, i.e., the replicon RNA cannot replicate in the host cell in the absence of the helper nucleic acid(s) encoding the alphavirus structural proteins.
As described in detail hereinbelow, the present invention provides improved alphavirus-based replicon systems that reduce the potential for replication-competent virus formation and that are suitable and/or advantageous for commercial-scale manufacture of vaccines or therapeutics comprising them. The present invention provides improved alphavirus RNA replicons and improved helpers for expressing alphavirus structural proteins.
In one embodiment of this invention, a series of “helper constructs,” i.e., recombinant DNA molecules that express the alphavirus structural proteins, is disclosed in which a single helper is constructed that will resolve itself into two separate molecules in vivo. Thus, the advantage of using a single helper in terms of ease of manufacturing and efficiency of production is preserved, while the advantages of a bipartite helper system are captured in the absence of employing a bipartite expression system. In one set of these embodiments, a DNA helper construct is used, while in a second set an RNA helper vector is used. In the case of the DNA helper constructs that do not employ alphaviral recognition signals for replication and transcription, the theoretical frequency of recombination is lower than the bipartite RNA helper systems that employ such signals.
In the preferred embodiments for the constructs of this invention, a promoter for directing transcription of RNA from DNA, i.e., a DNA dependent RNA polymerase, is employed. In the RNA helper embodiments, the promoter is utilized to synthesize RNA in an in vitro transcription reaction, and specific promoters suitable for this use include the SP6, T7, and T3 RNA polymerase promoters. In the DNA helper embodiments, the promoter functions within a cell to direct transcription of RNA. Potential promoters for in vivo transcription of the construct include eukaryotic promoters such as RNA polymerase II promoters, RNA polymerase III promoters, or viral promoters such as MMTV and MoSV LTR, SV40 early region, RSV or CMV. Many other suitable mammalian and viral promoters for the present invention are available in the art. Alternatively, DNA dependent RNA polymerase promoters from bacteria or bacteriophage, e.g., SP6, T7, and T3, may be employed for use in vivo, with the matching RNA polymerase being provided to the cell, either via a separate plasmid, RNA vector, or viral vector. In a specific embodiment, the matching RNA polymerase can be stably transformed into a helper cell line under the control of an inducible promoter. Constructs that function within a cell can function as autonomous plasmids transfected into the cell or they can be stably transformed into the genome. In a stably transformed cell line, the promoter may be an inducible promoter, so that the cell will only produce the RNA polymerase encoded by the stably transformed construct when the cell is exposed to the appropriate stimulus (inducer). The helper constructs are introduced into the stably transformed cell concomitantly with, prior to, or after exposure to the inducer, thereby effecting expression of the alphavirus structural proteins. Alternatively, constructs designed to function within a cell can be introduced into the cell via a viral vector, e.g., adenovirus, pox virus, adeno-associated virus, SV40, retrovirus, nodavirus, picornavirus, vesicular stomatitis virus, and baculoviruses with mammalian pol II promoters.
Once an RNA transcript (mRNA) encoding the helper or RNA replicon vectors of this invention is present in the helper cell (either via in vitro or in vivo approaches, as described above), it is translated to produce the encoded polypeptides or proteins. The initiation of translation from an mRNA involves a series of tightly regulated events that allow the recruitment of ribosomal subunits to the mRNA. Two distinct mechanisms have evolved in eukaryotic cells to initiate translation. In one of them, the methyl-7-G(5′)pppN structure present at the 5′ end of the mRNA, known as “cap,” is recognized by the initiation factor eIF4F, which is composed of eIF4E, eIF4G and eIF4A. Additionally, pre-initiation complex formation requires, among others, the concerted action of initiation factor eIF2, responsible for binding to the initiator tRNA-Met₁, and eIF3, which interacts with the 40S ribosomal subunit (reviewed in Hershey & Merrick, 2000).
In the alternative mechanism, translation initiation occurs internally on the transcript and is mediated by a cis-acting element, known as an internal ribosome entry site (IRES), that recruits the translational machinery to an internal initiation codon in the mRNA with the help of trans-acting factors (reviewed in Jackson, 2000). During many viral infections, as well as in other cellular stress conditions, changes in the phosphorylation state of eIF2, which lower the levels of the ternary complex eIF2-GTP-tRNA-Met.sub.1, results in overall inhibition of protein synthesis. Conversely, specific shut-off of cap-dependent initiation depends upon modification of eIF4F functionality (Thompson & Sarnow, 2000).
IRES elements bypass cap-dependent translation inhibition; thus the translation directed by an IRES is termed “cap-independent.” Hence, IRES-driven translation initiation prevails during many viral infections, for example picornaviral infection (Macejak & Sarnow, 1991). Under these circumstances, cap-dependent initiation is inhibited or severely compromised due to the presence of small amounts of functional eIF4F. This is caused by cleavage or loss of solubility of eIF4G (Gradi et al., 1998); 4E-BP dephosphorylation (Gingras et al., 1996) or poly(A)-binding protein (PABP) cleavage (Joachims et al., 1999).
IRES sequences have been found in numerous transcripts from viruses that infect vertebrate and invertebrate cells as well as in transcripts from vertebrate and invertebrate genes. Examples of IRES elements suitable for use in this invention include: viral IRES elements from Picornaviruses, e.g., poliovirus (PV), encephalomyocarditis virus (EMCV), foot-and-mouth disease virus (FMDV), from Flaviviruses, e.g., hepatitis C virus (HCV), from Pestiviruses, e.g., classical swine fever virus (CSFV), from Retroviruses, e.g., murine leukemia virus (MLV), from Lentiviruses, e.g., simian immunodeficiency virus (SIV), or cellular mRNA IRES elements such as those from translation initiation factors, e.g., eIF4G or DAP5, from Transcription factors, e.g., c-Myc (Yang and Sarnow, Nucleic Acids Research 25: 2800 2807 1997) or NF-κB-repressing factor (NRF), from growth factors, e.g., vascular endothelial growth factor (VEGF), fibroblast growth factor (FGF-2), platelet-derived growth factor B (PDGF B), from homeotic genes, e.g., Antennapedia, from survival proteins, e.g., X-Linked inhibitor of apoptosis (XIAP) or Apaf-1, or chaperones, e.g., the immunoglobulin heavy-chain binding protein BiP (reviewed in Martinez-Salas et al., 2001.)
Preferred IRES sequences that can be utilized in these embodiments are derived from: encephalomyocarditis virus (EMCV, accession # NC001479), cricket paralysis virus (accession # AF218039), Drosophila C virus accession # AF014388, Plautia stali intestine virus (accession # AB006531), Rhopalosiphum padi virus (accession # AF022937), Himetobi P virus (accession # AB017037), acute bee paralysis virus (accession # AF150629), Black queen cell virus (accession # AF183905), Triatoma virus (accession # AF178440), Acyrthosiphon pisu virus (accession # AF024514), infectious flacherie virus (accession # AB000906), and Sacbrood virus (accession # AF092924). In addition to the naturally occurring IRES elements listed above, synthetic IRES sequences, designed to mimic the function of naturally occurring IRES sequences, can also be used. In the embodiments in which an IRES is used for translation of the promoter driven constructs, the IRES may be an insect TRES or another non-mammalian IRES that is expressed in the cell line chosen for packaging of the recombinant alphavirus particles, but would not be expressed, or would be only weakly expressed, in the target host. In those embodiments comprising two IRES elements, the two elements may be the same or different.

The entire passage above is specifically incorporated herein by reference.

B. Other Viral Delivery Systems

i. Adenoviral Vectors

A particular method for delivery of the expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a coding region that has been inserted therein.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus et al., 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and/or late (L) regions of the genome contain different transcription units that are divided by the onset of viral DNA replication. The E1 region (E1A and/or E1B) encodes proteins responsible for the regulation of transcription of the viral genome and/or a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNA's issued from this promoter possess a 5′-tripartite leader (TPL) sequence which makes them preferred mRNA's for translation.

Recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from embryonic kidney cells by Ad5 DNA fragments and constitutively expresses E1 proteins (E1A and E1B; Graham et al., 1977). Since the E3 region is dispensable from the adenovirus genome (Jones and Shenk, 1978), the current adenovirus vectors, with the help of 293 cells, carry foreign DNA in either the E1, the D3 and/or both regions (Graham and Prevec, 1991). Recently, adenoviral vectors comprising deletions in the E4 region have been described (U.S. Pat. No. 5,670,488, incorporated herein by reference).

In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, and about 15% of the total length of the vector. More than 80% of the adenovirus viral genome remains in the vector backbone.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, and at least conditionally defective, the nature of the adenovirus vector is not believed to be crucial to the successful practice of the invention. The adenovirus may be of any of the 42 different known serotypes and subgroups A-F. Adenovirus type 5 of subgroup C is the preferred starting material in order to obtain the conditional replication-defective adenovirus vector for use in the present invention. This is because Adenovirus type 5 is a adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) and in the E4 region where a helper cell line and helper virus complements the E4 defect.

Adenovirus growth and manipulation is known to those of skill in the art, and exhibits broad host range in vitro and in vivo. This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus et al., 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993). Recombinant adenovirus and adeno-associated virus (see below) can both infect and transduce non-dividing primary cells.

ii. AAV Vectors

Adeno-associated virus (AAV) is an attractive vector system for use in the cell transduction of the present invention as it has a high frequency of integration and it can infect nondividing cells, thus making it useful for delivery of genes into cells, for example, in tissue culture (Muzyczka, 1992) and in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Yoder et al., 1994; Zhou et al., 1994; Hermonat and Muzyczka, 1984; Tratschin et al., 1985; McLaughlin et al., 1988) and genes involved in various diseases (Flotte et al., 1992; Ohi et al., 1990; Walsh et al., 1994; Wei et al., 1994). Recently, an AAV vector has been approved for phase I trials for the treatment of cystic fibrosis.

AAV is a dependent parvovirus in that it requires coinfection with another virus (either adenovirus or a member of the herpesvirus family) to undergo a productive infection in cultured cells (Muzyczka, 1992). In the absence of coinfection with helper virus, the wild type AAV genome integrates through its ends into chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte et al., 1995).

iii. Retroviral Vectors

Retroviruses have promise as gene delivery vectors due to their ability to integrate their genes into the host genome, transferring a large amount of foreign genetic material, infecting a broad spectrum of species or cell types and of being packaged in special cell-lines (Miller, 1992).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Gene delivery using second generation retroviral vectors has been reported. Kasahara et al. (1994) prepared an engineered variant of the Moloney murine leukemia virus, that normally infects only mouse cells, and modified an envelope protein so that the virus specifically bound to, and infected, cells bearing the erythropoietin (EPO) receptor. This was achieved by inserting a portion of the EPO sequence into an envelope protein to create a chimeric protein with a new binding specificity.

iv. Other Viral Vectors

Other viral vectors may be employed as expression constructs in the present invention. Vectors derived from viruses such as vaccinia virus (Ridgeway, 1988; Baichwal and Sugden, 1986; Coupar et al., 1988), sindbis virus, cytomegalovirus or herpes simplex virus may be employed. They offer several attractive features for various cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In certain further embodiments, the gene therapy vector will be HSV. A factor that makes HSV an attractive vector is the size and organization of the genome. Because HSV is large, incorporation of multiple genes and expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations. HSV also is relatively easy to manipulate and can be grown to high titers. Thus, delivery is less of a problem, both in terms of volumes needed to attain sufficient MOI and in a lessened need for repeat dosings.

v. Modified Viruses

In still further embodiments of the present invention, the nucleic acids to be delivered are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein or against a specific cell receptor were used. The antibodies were coupled via the biotin components by using streptavidin (Roux et al., 1989). Using antibodies against major histocompatibility complex class I or class II antigens, they demonstrated the infection of a variety of cells that bore those surface antigens with an ecotropic virus in vitro (Roux et al., 1989).

C. Non-Viral Delivery Systems

A variety of non-viral delivery systems are available. Many of these rely on lipid formulations generically referred to as “liposomes” or “nanoparticles.” Liposomes are defined a spherical vesicle composed of a bilayer membrane. In biology, this specifically refers to a membrane composed of a phospholipid and cholesterol bilayer. Liposomes can be composed of naturally-derived phospholipids with mixed lipid chains (e.g., egg phosphatidylethanolamine), or of pure surfactant components like DOPE (dioleoylphosphatidylethanolamine). Liposomes, usually but not by definition, contain a core of aqueous solution; lipid spheres that contain no aqueous material are called micelles, however, reverse micelles can be made to encompass an aqueous environment.

Liposomes are used for drug delivery due to their unique properties. A liposome encapsulates a region on aqueous solution inside a hydrophobic membrane; dissolved hydrophilic solutes can not readily pass through the lipids. Hydrophobic chemicals can be dissolved into the membrane, and in this way liposome can carry both hydrophobic molecules and hydrophilic molecules. To deliver the molecules to sites of action, the lipid bilayer can fuse with other bilayers such as the cell membrane, thus delivering the liposome contents. By making liposomes in a solution of DNA or drugs (which would normally be unable to diffuse through the membrane) they can be (indiscriminately) delivered past the lipid bilayer.

Liposomes can also be designed to deliver drugs in other ways. Liposomes that contain low (or high) pH can be constructed such that dissolved aqueous drugs will be charged in solution (i.e., the pH is outside the drug's pI range). As the pH naturally neutralizes within the liposome (protons can pass through some membranes), the drug will also be neutralized, allowing it to freely pass through a membrane. These liposomes work to deliver drug by diffusion rather than by direct cell fusion. Another strategy for liposome drug delivery is to target endocytosis events. Liposomes can be made in a particular size range that makes them viable targets for natural macrophage phagocytosis. These liposomes may be digested while in the macrophage's phagosome, thus releasing its drug. Liposomes can also be decorated with opsonins and ligands to activate endocytosis in other cell types. The use of liposomes for transformation or transfection of DNA into a host cell is known as lipofection.

Liposomes can be created by sonicating phospholipids in water. Low shear rates create multilamellar liposomes, which have many layers like an onion. Continued high-shear sonication tends to form smaller unilamellar liposomes. In this technique, the liposome contents are the same as the contents of the aqueous phase. Sonication is generally considered a “gross” method of preparation, and newer methods such as extrusion are employed to produce materials for human use.

Lipid formulations have been used for efficient of gene transfer in vivo (Smyth-Templeton et al., 1997; WO 98/07408). A lipid formulation composed of an equimolar ratio of 1,2-bis(oleoyloxy)-3-(trimethyl ammonio)propane (DOTAP) and cholesterol significantly enhances systemic in vivo gene transfer, approximately 150-fold. The DOTAP:cholesterol lipid formulation is said to form a unique structure termed a “sandwich liposome.” This formulation is reported to “sandwich” DNA between an invaginated bi-layer or “vase” structure. Beneficial characteristics of these lipid structures include a positive colloidal stabilization by cholesterol, two dimensional DNA packing and increased serum stability.

A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof.

A neutral fat may comprise a glycerol and a fatty acid. A typical glycerol is a three carbon alcohol. A fatty acid generally is a molecule comprising a carbon chain with an acidic moeity (e.g., carboxylic acid) at an end of the chain. A fatty acid comprising only single bonds in its carbon chain is called saturated, while a fatty acid comprising at least one double bond in its chain is called unsaturated. Specific fatty acids include, but are not limited to, linoleic acid, oleic acid, palmitic acid, linolenic acid, stearic acid, lauric acid, myristic acid, arachidic acid, palmitoleic acid, arachidonic acid ricinoleic acid, tuberculosteric acid, lactobacillic acid. An acidic group of one or more fatty acids is covalently bonded to one or more hydroxyl groups of a glycerol. Thus, a monoglyceride comprises a glycerol and one fatty acid, a diglyceride comprises a glycerol and two fatty acids, and a triglyceride comprises a glycerol and three fatty acids.

A phospholipid generally comprises either glycerol or an sphingosine moiety, an ionic phosphate group to produce an amphipathic compound, and one or more fatty acids. Types of phospholipids include, for example, phophoglycerides, wherein a phosphate group is linked to the first carbon of glycerol of a diglyceride, and sphingophospholipids (e.g., sphingomyelin), wherein a phosphate group is esterified to a sphingosine amino alcohol. Another example of a sphingophospholipid is a sulfatide, which comprises an ionic sulfate group that makes the molecule amphipathic. A phospholipid may, of course, comprise further chemical groups, such as for example, an alcohol attached to the phosphate group. Examples of such alcohol groups include serine, ethanolamine, choline, glycerol and inositol. Thus, specific phosphoglycerides include a phosphatidyl serine, a phosphatidyl ethanolamine, a phosphatidyl choline, a phosphatidyl glycerol or a phosphotidyl inositol. Other phospholipids include a phosphatidic acid or a diacetyl phosphate. In one aspect, a phosphatidylcholine comprises a dioleoylphosphatidylcholine (a.k.a. cardiolipin), an egg phosphatidylcholine, a dipalmitoyl phosphalidycholine, a monomyristoyl phosphatidylcholine, a monopalmitoyl phosphatidylcholine, a monostearoyl phosphatidylcholine, a monooleoyl phosphatidylcholine, a dibutroyl phosphatidylcholine, a divaleroyl phosphatidylcholine, a dicaproyl phosphatidylcholine, a diheptanoyl phosphatidylcholine, a dicapryloyl phosphatidylcholine or a distearoyl phosphatidylcholine.

A glycolipid is related to a sphinogophospholipid, but comprises a carbohydrate group rather than a phosphate group attached to a primary hydroxyl group of the sphingosine. A type of glycolipid called a cerebroside comprises one sugar group (e.g., a glucose or galactose) attached to the primary hydroxyl group. Another example of a glycolipid is a ganglioside (e.g., a monosialoganglioside, a GM1), which comprises about 2, about 3, about 4, about 5, about 6, to about 7 or so sugar groups, that may be in a branched chain, attached to the primary hydroxyl group. In other embodiments, the glycolipid is a ceramide (e.g., lactosylceramide).

A steroid is a four-membered ring system derivative of a phenanthrene. Steroids often possess regulatory functions in cells, tissues and organisms, and include, for example, hormones and related compounds in the progestagen (e.g., progesterone), glucocoricoid (e.g., cortisol), mineralocorticoid (e.g., aldosterone), androgen (e.g., testosterone) and estrogen (e.g., estrone) families. Cholesterol is another example of a steroid, and generally serves structural rather than regulatory functions. Vitamin D is another example of a sterol, and is involved in calcium absorption from the intestine.

A terpene is a lipid comprising one or more five carbon isoprene groups. Terpenes have various biological functions, and include, for example, vitamin A, coenyzme Q and carotenoids (e.g., lycopene and β-carotene).

In certain embodiments, a lipid component of a composition is uncharged or primarily uncharged. In one embodiment, a lipid component of a composition comprises one or more neutral lipids. In another aspect, a lipid component of a composition may be substantially free of anionic and cationic lipids, such as certain phospholipids (e.g., phosphatidyl choline) and cholesterol. In certain aspects, a lipid component of an uncharged or primarily uncharged lipid composition comprises about 95%, about 96%, about 97%, about 98%, about 99% or 100% lipids without a charge, substantially uncharged lipid(s), and/or a lipid mixture with equal numbers of positive and negative charges.

In other aspects, a lipid composition may be charged. For example, charged phospholipids may be used for preparing a lipid composition according to the present invention and can carry a net positive charge or a net negative charge. In a non-limiting example, diacetyl phosphate can be employed to confer a negative charge on the lipid composition, and stearylamine can be used to confer a positive charge on the lipid composition.

A liposome used according to the present invention can be made by different methods, as would be known to one of ordinary skill in the art. Phospholipids can form a variety of structures other than liposomes when dispersed in water, depending on the molar ratio of lipid to water. For example, a phospholipid (Avanti Polar Lipids, Alabaster, AL), such as for example the neutral phospholipid dioleoylphosphatidylcholine (DOPC), is dissolved in tert-butanol. The lipid(s) is then mixed with the active agent(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline. The average diameter of the particles obtained using Tween 20 for encapsulating the active agent(s) is about 0.7 to about 1.0 μm in diameter.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time. Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In other alternative methods, liposomes can be prepared in accordance with other known laboratory procedures (e.g., see Bangham et al., 1965; Gregoriadis, 1979; Deamer and Uster 1983, Szoka and Papahadjopoulos, 1978, each incorporated herein by reference in relevant part). These methods differ in their respective abilities to entrap aqueous material and their respective aqueous space-to-lipid ratios.

The dried lipids or lyophilized liposomes prepared as described above may be dehydrated and reconstituted in a solution of inhibitory peptide and diluted to an appropriate concentration with an suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000×g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

The size of a liposome varies depending on the method of synthesis. Liposomes in the present invention can be a variety of sizes. In certain embodiments, the liposomes are small, e.g., less than about 100 nm, about 90 nm, about 80 nm, about 70 nm, about 60 nm, or less than about 50 nm in external diameter. In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040; U.K. Patent Application GB 2193095 A; Mayer et al., 1986; Hope et al., 1985; Mayhew et al. 1987; Mayhew et al., 1984; Cheng et al., 1987; and Liposome Technology, 1984, each incorporated herein by reference).

A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

The production of lipid formulations often is accomplished by sonication or serial extrusion of liposomal mixtures after (I) reverse phase evaporation (II) dehydration-rehydration (III) detergent dialysis and (IV) thin film hydration. In one aspect, a contemplated method for preparing liposomes in certain embodiments is heating sonicating, and sequential extrusion of the lipids through filters or membranes of decreasing pore size, thereby resulting in the formation of small, stable liposome structures. This preparation produces liposomes only of appropriate and uniform size, which are structurally stable and produce maximal activity. Such techniques are well-known to those of skill in the art (see, for example Martin, 1990).

Alternate methods of preparing lipid-based formulations for nucleic acid delivery are described (WO 99/18933). In another liposome formulation, an amphipathic vehicle called a solvent dilution microcarrier (SDMC) enables integration of particular molecules into the bi-layer of the lipid vehicle (U.S. Pat. No. 5,879,703). The SDMCs can be used to deliver lipopolysaccharides, polypeptides, nucleic acids and the like. Of course, any other methods of liposome preparation can be used by the skilled artisan to obtain a desired liposome formulation in the present invention.

D. Gene Gun

The “gene gun” or Biolistic Particle Delivery System was originally designed for injecting plant cell with heterologous genetic information. The payload is an elemental particle of a heavy metal coated with DNA. This technique is often simply referred to as biolistics. This device is able to transform almost any type of cell, including plants, and is not limited to genetic material of the nucleus—it can also transform organelles, including plastids. Other heavy metals such as gold and silver are also used. Gold may be favored because it has better uniformity than tungsten and tungsten can be toxic to cells, but its use may be limited due to availability and cost.

Gene guns have so far mostly been applied to plants transformation. However, there it can be usee in animals and humans as well. For example, gene guns have been used to deliver DNA vaccines to experimental animals. The delivery of plasmids into rat neurons through the use of a gene gun, specifically DRG neurons, is also used as a pharmacological precursor in studying the effects of neurodegenerative diseases such as Alzheimer's Disease.

### III. FORMULATIONS AND ADMINISTRATION

A. Formulations

The phrases “pharmaceutically acceptable” or “pharmacologically acceptable” refer to molecular entities and compositions that do not produce an adverse, allergic, or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents, and the like. The use of such reagents for pharmaceutical substances is well known in the art. Except insofar as any conventional agent is incompatible with the active ingredients, its use in the therapeutic compositions is contemplated. Supplementary active ingredients, such as adjuvants or biological response modifiers, can also be incorporated into the administration.

Also contemplated are pharmaceutically acceptable salts, include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intradermal, and intraperitoneal administration. In this connection, sterile aqueous media that can be employed will be known to those of skill in the art in light of the present disclosure.

An effective amount of the compositions of the present invention is determined based on the intended goal of generating an immune response. The term “unit dose” or “dosage” refers to physically discrete units suitable for use in a subject, each unit containing a predetermined-quantity of the composition calculated to produce the desired immune responses, discussed above, in association with its administration, i.e., the appropriate route and regimen. The quantity to be administered, both according to number of treatments and unit dose, depends on the protection desired.

For viral vectors, one generally will prepare a viral vector stock of high titer. Depending on the titer attainable, one will deliver 1 to 100, 10 to 50, 100-1000, or up to 1×10⁴, 1×10⁵, 1×10⁶, 1×10⁷, 1×10⁸, 1×10⁹, 1×10¹⁰, 1×10¹¹, 1×10¹², 1×10¹³ or 1×10¹⁴ particles to the patient. A similar number of VLPs may be used as well. Formulation as a pharmaceutically acceptable composition is discussed below above.

B. Vaccination Protocols

The vaccines of the present invention—DNA constructs that express the structural paramyxovirus proteins, or the VLPs themselves—can be formulated for parenteral administration, e.g., formulated for injection via the intradermal, intravenous, intramuscular, subcutaneous, or even intraperitoneal routes. Administration by the intradermal and intramuscular routes are specifically contemplated. The vaccine could alternatively be administered by a topical route directly to the mucosa, for example by nasal drops, inhalation, or by nebulizer.

Some variation in dosage and regimen will necessarily occur depending on the age and possibly medical condition of the subject being treated. The person responsible for administration will, in any event, determine the appropriate dose for the individual subject. In many instances, it will be desirable to have several or multiple administrations of the vaccine. The compositions of the invention may be administered 1, 2, 3, 4, 5, 6, 7, 8, 9, 10, or more times. The administrations will normally be at from one to twelve week intervals, more usually from one to four week intervals. Periodic re-administration will be desirable with recurrent exposure to the pathogen.

It may also be useful to provide, in certain embodiments, an adjuvant. Adjuvants are pharmacological or immunological agents that modify the effect of other agents (i.e., vaccines) while having few if any direct effects when given by themselves. In this sense, they are roughly analogous to chemical catalysts. Types of adjuvants include Freund's (complete and incomplete), saponins (e.g., QuilA, QS21), muramyl dipeptides and derivatives (MTP-PE), copolymers, ISCOMS, cytokines, and oligonucleotides.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

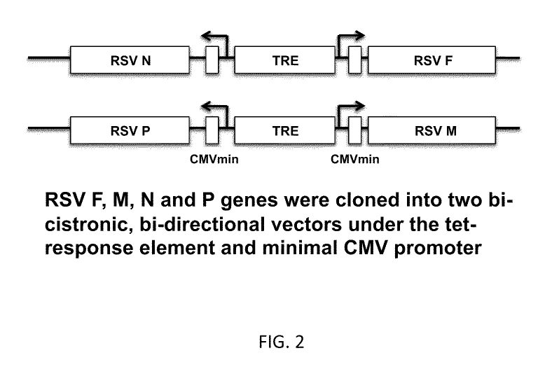

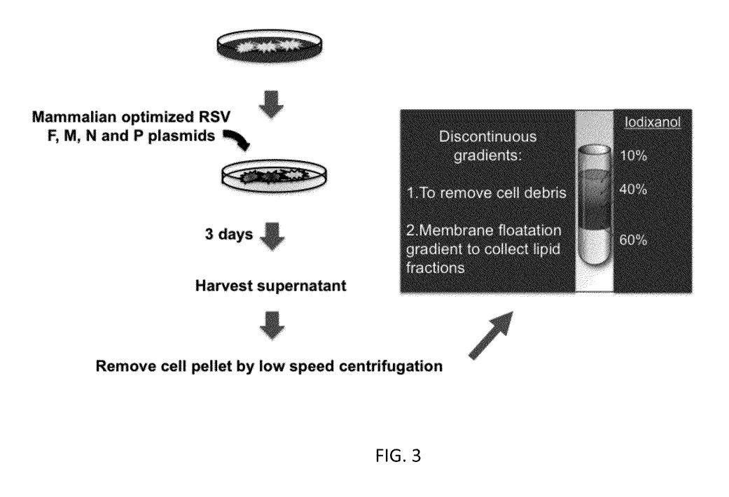

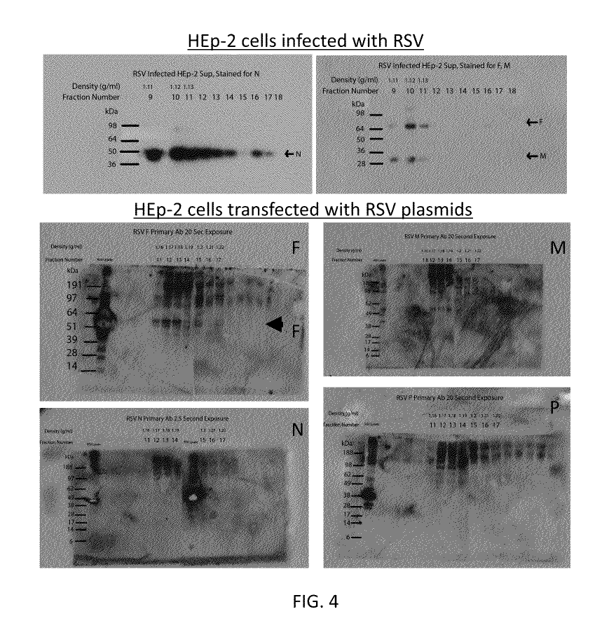

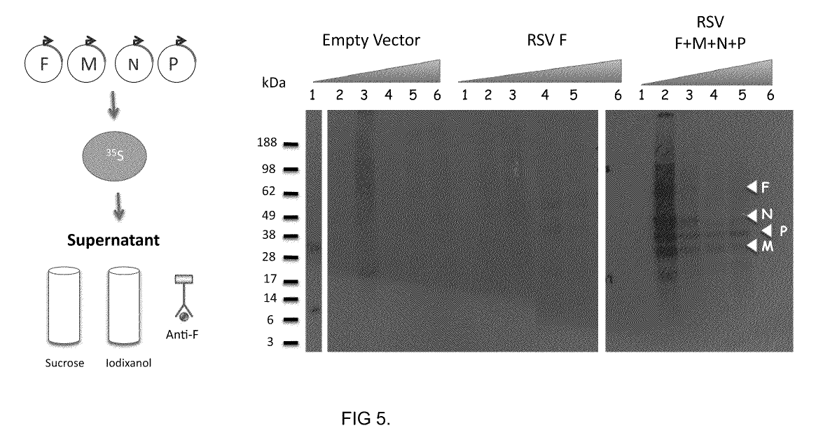

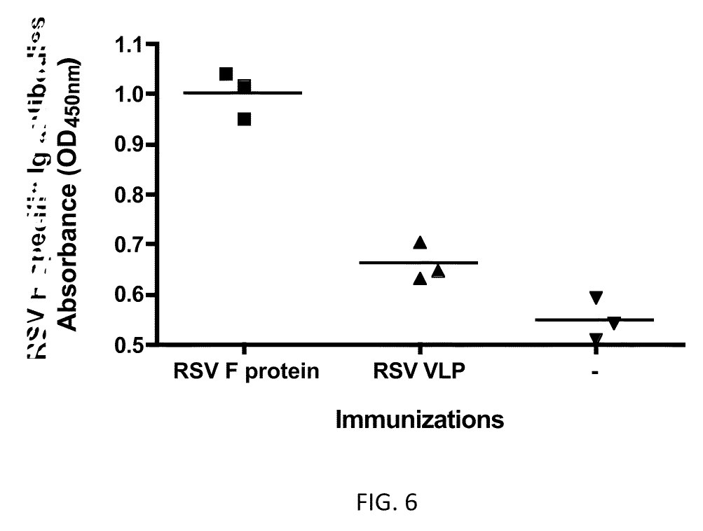

The inventors sought to develop and test the use of RSV virus-like particles (VLPs) as a novel preventative vaccine candidate. RSV VLPs were produced by transfecting cultured cells with four plasmids encoding cDNAs of the viral proteins F (fusion), M (matrix), N (nucleoprotein), and P (phosphoprotein). Expressed VLPs were then harvested from cell supernatants collected by a membrane flotation isolation procedure. The isolation of VLPs was optimized by analyzing each collected fraction by western blot to detect the presence of each of the four viral proteins. Under optimal expression and isolation conditions, all four viral proteins were detected by western blot analysis suggesting the formation of VLPs. The expression process was performed with different combinations of three of the plasmids to confirm that the four selected proteins constitute the minimal requirement for VLP assembly in vivo. The ability our RSV VLPs to generating an effective immune response was determined in BALB/c mice. A remarkably small dose of unadjuvented VLPs (500 ng) induced high serum titers of RSV-specific antibodies in vaccinated animals, as detected by ELISA. These encouraging results suggest a VLP vaccination approach may be an innovative new approach to vaccinating against RSV. See FIGS. 2-6.

These data show that RSV antigenic particles (VLPs) are formed in mammalian cell lines transfected with genes encoding the viral proteins F, M, N, and P. Initial animal studies showed increased levels of RSV F-specific antibodies in VLP-immunized BALB/c mice. Ongoing work is underway to extend these studies to the generation of immune responses to the particles in vivo.

Next, the inventors sought to explore the immune mechanisms of protection, by passively transferring antibodies from immunized animals and depleting T cell subsets. They showed that a single dose of alphavirus replicon particles vaccine encoding RSV F genes induced robust antibody responses independent of the immune status of the vaccinees. A single dose was used because it is more likely to be suited for RSV vaccination in young infants due to the narrow time window between vaccination and possible natural infection. The inventors showed that, unlike live RSV vaccination, VRP-RSV.F induced similar antibody neutralization responses against RSV F in immune animals compared to naïve animals. Delivering vaccine antigen by a heterologous viral vector (VRP) might provide temporary “shelter” for RSV antigens before they are recognized and neutralized by circulating RSV antibodies.

A recent report has also documented that VRP-based dengue vaccine could overcome maternal antibody interference in weanling mice (White, 2007) and supports the idea of unique properties of VRP compared to other viral-based vectors. Enumeration of RSV F-specific B cells in the lungs and mediastinal lymph nodes revealed a similar trend and correlated to the antibody data. RSV F-specific IgG secreting B cells were reduced dramatically in immune animals vaccinated with live RSV when challenged with RSV compared to naïve animals. A surprising finding is that intramuscular injected VRP did not induce a strong B cell response in the mucosal compartment, possibly due to the early sampling time (3 days), although there are detectable responses in most of the vaccinated animals.

In an antibody depletion study, the inventors showed that RSV F-specific CD8+ T cells are contributors to RSV resolution in VRP-vaccinated mice lacking a majority of B cells. CD4+ T cells did not play a role in this case because the major T cell epitope on F protein in BALB/c mice is restricted to an MHC class I molecules (F₈₅₋₉₃). It is possible that immunization in humans by VRP would induce both RSV F-specific CD4+ and CD8+ T cells, as was demonstrated in human peripheral blood mononuclear cells using recombinant modified vaccinia virus Ankara encoding F protein. They also showed that RSV F-specific T cell responses were not statistically different in both the systemic and mucosal compartments of vaccinated animals after RSV challenge. Surprisingly, there was a reduction in IFN-γ secreting T cell response in the spleen in VRP vaccinated mice after immunization. There has been a report that showed passively acquired antibodies to RSV could impair secondary CTL in neonatal mouse (Bangham, 1986), but this was not shown to be the case in other studies. The T cell response in the spleen, however, rebounded to a higher level after RSV challenge in immune animals. It has been shown recently that antigen-specific CD8+ T cells could travel to mucosal surfaces following intramuscular adenovirus vaccination (Kaufman, 2008). The numbers of RSV F-specific T cells mirrored those in naïve animals after challenge, signifying that VRP primes for a robust systemic T cell response upon natural infection.

The inventors also examined the cytolytic gene expression of RSV F-specific CD8+ T cells in live RSV vaccinated and VRP vaccinated animals. Gene expression of antigen-specific CD8+ T cells has been studied for various viruses including LCMV (Grayson, 2001). Cytolytic gene expression depends of types of T cells (effector versus memory), stimulation history (Masopust, 2006) and length of activation. The inventors showed that antigen specific T cells induced by VRP vaccination had about 2-fold increase in cytolytic gene expression including perforin and granzyme A as compared to those induced by RSV infection. Although the significance of increase in cytolytic activity is not known at this point, it nonetheless demonstrates a different pathway of activation by VRP vaccination than RSV infection.

Thus, the inventors have successfully demonstrated that VRP encoding RSV F primed and induced robust humoral and cellular responses in vaccinated animals against RSV in the presence of pre-existing neutralizing antibodies. When delivered intramuscularly, this vaccine induced mucosal T cell responses similar to those delivered intranasally. In addition, the effector function of RSV F-specific CD8+ T cells was enhanced by VRP vaccination.

The experimental plan will be to immunize animals with various combinations of RSV protein encoding plasmids to determine if simultaneous immunization with RSV F, M, N and P leads to in vivo virus-like particle formation, high immunogenicity and complete protective efficacy. Gene delivery will be attempted through several strategies, including DNA immunization with needles (IM and ID routes), DNA immunization with gene gun, and viral vector delivery (bicistronic alphavirus replicons). The inventors can easily determine if the strategy works by designing well-controlled experiments. For example, the principal strategy of interest is to immunize with the four plasmids, but they will also immunize with single plasmids and combinations of two or three plasmids. The hypothesis to be tested is that the four-gene immunization will lead to the highest immunogenicity. Immunogenicity and efficacy are easily measured with assays that are in hand, including RSV F antibody ELISA, virus neutralizing antibody assay, RSV-specific CTL assays and tetramer assays, and most importantly, titer of virus after wild-type challenge. All of these assays are well-described in the inventors' previous publications. If the strategy shows that the four-gene strategy is the most immunogenic and efficacious, the inventors plan to conduct more mechanistic experiments. First, they will attempt to show directly that virus filament formation occurs in vivo by immunohistopathologic examination of the immunization site in vaccinated animals, using immunofluorescence and electron microcopy studies. The gene gun method will be especially helpful here, since the delivery beads can be detected by EM.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VI. REFERENCES

The following references, to the extent that they provide exemplary procedural or other; details supplementary to those set forth herein, are specifically incorporated herein by reference.
U.S. Pat. No. 4,797,368
U.S. Pat. No. 4,728,578
U.S. Pat. No. 4,728,575
U.S. Pat. No. 4,737,323
U.S. Pat. No. 4,533,254
U.S. Pat. No. 4,162,282
U.S. Pat. No. 4,310,505
U.S. Pat. No. 4,921,706
U.S. Pat. No. 5,139,941
U.S. Pat. No. 5,185,440
U.S. Pat. No. 5,505,947
U.S. Pat. No. 5,643,576
U.S. Pat. No. 5,792,462
U.S. Pat. No. 5,879,703
U.S. Pat. No. 6,156,558
U.S. Pat. No. 6,521,235
U.S. Pat. No. 6,531,135
U.S. Pat. No. 6,541,010
U.S. Pat. No. 6,738,939
U.S. Pat. No. 7,045,335
U.S. Pat. No. 7,078,218
U.S. Pat. No. 7,045,335
U.S. Patent Pubn. 2002/014975 A1
Baichwal and Sugden, In: Gene Transfer, Kucherlapati (ed.), New York, Plenum Press, 117-148, 1986.
Balasuriya et al., Vaccine, 20(11-12):1609-17, 2002.
Bangham, et al., J. Mol. Biol., 13:238-252, 1965.
Bangham, CRM Immunology, 59:37-41, 1986.
Bastien et al., J. Clin. Microbiol., 42:3532-7, 2004.
Bastien et al., Virus Res., 93:51-62, 2003.
Biacchesi et al., J. Virol., 78:12877-87, 2004.
Biacchesi et al, Virology, 315:1-9, 2003.
Boivin et al., Emerg. Infect. Dis., 10:1154-7, 2004.
Boivin et al., J. Infect. Dis., 186:1330-4, 2002.
Bukreyev et al., J. Virol., 80:5854-61, 2006.
Burkhard et al., Vaccine, 21(3-4):258-68, 2002.
Cannon et al., J. Exp. Med., 168:1163-8, 1988.
Chang et al., Hepatology, 14:134 A, 1991.
Chen et al., J. Immunol., 169(6):3208-16, 2002.
Clark et al., Hum. Gene Ther., 6(10):1329-1341, 1995.
Coffin, Retroviridae and Their Replication. In: Virology, Fields et al., eds., Raven Press, New York, pp. 1437-1500, 1990.
Connors et al., J. Virol., 65(3):1634-7, 1991.
Connors et al., J. Virol., 65:1634-7, 1991.
Connors et al., J. Virol., 66(2):1277-81, 1992.
Connors et al., Virology, 208(2):478-84, 1995.
Corvaisier et al., Res. Virol., 144(2):141-50, 1993.
Couch et al., Am. Rev. Resp. Dis., 88:394-403, 1963.
Coupar et al., Gene, 68:1-10, 1988.
Crowe et al., J. Infect. Dis., 173(4):829-39, 1996a.
Crowe et al., J. Infect. Dis., 177(4):1073-6, 1998.
Crowe et al., Vaccine, 11(14):1395-404, 1993.
Crowe et al., Vaccine, 12(8):691-9, 1994b.
Crowe et al., Vaccine, 12(9):783-90, 1994a.
Crowe et al., Vaccine, 13(9):847-55, 1995.
Crowe et al., Virus Genes, 13(3):269-73, 1996b.
Cseke et al., J. Virol., 81:698-707, 2007.
Davis et al., IUBMB Life, 53(4-5):209-11, 2002.
Davis et al., J. Virol., 70(6):3781-7, 1996.
Deamer and P. Uster, “Liposome Preparation: Methods and Mechanisms,” in Liposomes (M. Ostro, ed.), Marcel Dekker, Inc., New York (1983), pp. 27-52.
Durbin et al., J. Immunol., 168(6):2944-52, 2002.
Ebihara et al., J. Clin. Microbiol., 42:5944-6, 2004.
EPO 0 273 085
Esper et al., J. Infect. Dis., 189:1388-96, 2004.
Falsey et al., J. Infect. Dis., 187:785-90, 2003.
Firestone et al., Virology, 225(2):419-22, 1996.
Flotte et al., Am. J. Respir. Cell. Mol. Biol., 7(3):349-356, 1992.
Flotte et al., Gene Ther., 2(1):29-37, 1995.
Flotte et al., Proc. Nat'l Acad. Sci. USA, 90(22):10613-7, 1993.
Fraley and Fomari-Kaplan, Proc. Nat'l. Acad. Sci. USA 76:3348-3352, 1979.
Ghosh and Bachhawat, “Targeting of liposomes to hepatocytes,” In. Wu G. Wu C ed., Liver diseases, targeted diagnosis and therapy using specific receptors and ligands, New York: Marel Dekker, pp. 87-104, 1991.
Ghosh-Choudhury et al., EMBO J, 6:1733-1739, 1987.
Gingras et al., Proc. Natl. Acad. Sci. USA, 93:5578 5583, 1996.
Gipson et al., Vaccine, 21(25-26):3875-84, 2003.
Gomez-Foix et al., J. Biol. Chem., 267:25129-25134, 1992.
Gradi et al., Proc. Natl. Acad. Sci. USA, 95:11089 11094, 1998.
Graham et al., J. Gen. Virol., 36:59-72, 1977.
Graham and Prevec, Biotechnology, 20:363-390, 1992.
Graham and Prevec, In: Methods in Molecular Biology. Gene Transfer and Expression Protocol, Murray (Ed.), Humana Press, Clifton, N.J., 7:109-128, 1991.
Grayson, J. Immunol., 166:795-99, 2001.
Gregoriadis G. and Davis C., Biochem Biophys Res Commun., 89(4):1287-1293, 1979.
Gregoriadis, DRUG CARRIERS IN BIOLOGY AND MEDICINE, G. Gregoriadis (ed.), 1979, pp. 287-341.
Grunhaus et al., Seminar in Virology, 200(2):535-546, 1992.
Haller et al., J. Gen. Virol., 84(Pt 8):2153-62, 2003.
Hancock et al., J. Virol., 70(11):7783-91, 1996.
Harrington et al., J. Virol., 76(2):730-42, 2002.
Herd et al. J. Virol., 80:2034-44, 2006.
Hermonat and Muzycska, Proc. Nat'l Acad. Sci. USA, 81:6466-6470, 1984.
Hersdorffer et al., DNA Cell Biol., 9:713-723, 1990.
Hershey & Merrick, In: Translational Control of Gene Expression, 33-88, Cold Spring Harbor Laboratory Press, NY, 2000.
Herz and Gerard, Proc. Nat'l Acad. Sci. USA, 90:2812-2816, 1993.
Hevey et al., Virology, 251(1):28-37, 1998.
Homa et al., J. Gen. Virol., 74 (Pt 9):1995-9, 1993.
Horwich et al., Virol., 64:642-650, 1990.
Hsu et al., J. Infect. Dis., 166(4):769-75, 1992.
Hsu et al., Vaccine, 13(5):509-15, 1995.
Huckriede et al., Vaccine, 22(9-10):1104-13, 2004.
Jackson, In: Translational Control of Gene Expression, 127-184, Cold Spring Harbor Laboratory Press, NY, 2000.
Joachims et al., J. Virol., 73:718 727, 1999.
Johnson et al., J. Virol., 72(4):2871-80, 1998.
Johnston et al., Vaccine, 23(42):4969-79, 2005.
Jones and Shenk, Cell, 13:181-188, 1978.
Juhasz et al., J. Virol., 71(8):5814-9, 1997.
Kahn et al., J. Virol., 75(22):11079-87, 2001.
Kaneda et al., Science, 243:375-378, 1989.
Kaneda et al., J Biol. Chem., 264(21):12126-12129, 1989.
Kapikian et al., Am. J. Epidemiol., 89(4):405-21, 1969.
Kaplitt et al., Nat. Genet., 8(2):148-54, 1994.
Karlsson et al., EMBO J, 5:2377-2385, 1986.
Karron et al., J. Infect. Dis., 176(6):1428-36, 1997.
Karron et al., J. Infect. Dis., 191(7):1093-104, 2005.
Kasahara et al., Science, 266(5189):1373-6, 1994.
Kato et al., J Biol. Chem., 266(6):3361-3364, 1991.
Kaufman, J. Immunol., 181:4188-98, 2008.
Kim et al., Am. J. Epidemiol., 89(4):422-34, 1969.
Kotin et al., Proc. Nat'l Acad. Sci. USA, 1990 March; 87(6):2211-5, 1990.
Kulkarni et al., J. Virol., 67(2):1044-9, 1993.
LaFace et al., Virology, 162(2):483-486, 1988.
Lee et al., J. Infect. Dis., 185(8):1192-6, 2002.
Laughlin et al., J. Virol., 60(2):515-524, 1986.
Le Gal La Salle et al., Science, 259:988-990, 1993.
Lebkowski et al., Mol. Cell. Biol., 8(10):3988-3996, 1988.
Levrero et al., Gene, 101:195-202, 1991.
MacDonald et al., J. Virol., 74(2):914-22, 2000.
Macejak and Sarnow, Nature, 353:90-94, 1991.
McLaughlin et al., J. Virol., 62(6):1963-1973, 1988.
Mann et al., Cell, 33:153-159, 1983.
Markowitz et al., J. Virol., 62:1120-1124, 1988.
Martinez-Salas et al., J. Gen. Virol., 82:973 984, 2001.
Martinez-Sobrido et al., J. Virol., 80(3):1130-9, 2006.
Masopust, J. Immunol., 177:831-39, 2006.
McAdam et al., J. Infect. Dis., 190:20-6, 2004.
Melendi et al., J. Virol., 2007. [epub]
Miller et al., Am. J. Clin. Oncol., 15(3):216-221, 1992.
Moghaddam et al., Nat. Med., 12(8):905-7, 2006.
Moran et al. J. Immunol., 175(5):3431-8, 2005.
Murphy et al., J. Clin. Microbiol., 24(2):197-202, 1986.
Murphy et al., J. Virol., 62(10):3907-10, 1988.
Nicolas and Rubinstein, In: Vectors. A survey of molecular cloning vectors and their uses, Rodriguez and Denhardt, eds., Stoneham: Butterworth, pp. 494-513, 1988.
Nicolau and Sene, Biochem. Biophys. Acta, 721:185-190, 1982.
Nicolau et al., Methods Enzymol., 149:157-176, 1987.
Ohi et al., Gene, 89(2):279-282, 1990.
Olmsted et al., Proc. Natl. Acad. Sci. USA, 83(19):7462-6, 1986.
Osterhaus and Fouchier, Lancet., 361:890-1, 2003.
Paskind et al., Virology, 67:242-248, 1975.
PCT/US85/01161
PCT/US89/05040
Peebles et al., J Allergy Clin. Immunol., 113(1 Suppl):S15-8, 2004.
Pelletier et al., Emerg. Infect. Dis., 8:976-8, 2002.
Pelletier and Sonenberg, Nature, 334:320-325, 1988.
Perales et al., Proc. Natl. Acad. Sci. USA, 91:4086-4090, 1994.
Peroulis et al., Arch. Virol., 144(1):107-16, 1999.
Plotnicky-Gilquin et al., J. Virol., 76:10203-10, 2002.
Polack et al. Proc. Natl. Acad. Sci. USA, 102:8996-9001, 2005.
Power et al., Virology, 230(2):155-66, 1997.
Prince et al. J. Virol., 55:517-20, 1985.
Prince et al., J. Virol., 57(3):721-8, 1986.
Prince et al., Virus Res., 3(3):193-206, 1985.
Pushko et al., J. Virol., 75(23):11677-85, 2001.
Pushko et al., Virology, 239(2):389-401, 1997.
Pushko et al., Virology, 239:389-401, 1997.
Ragot et al., Nature, 361:647-650, 1993.
Rich et al., Hum. Gene Ther., 4:461-476, 1993.
Rosenfeld et al., Cell, 68:143-155, 1992.
Rosenfeld et al., Science, 252:431-434, 1991.
Roux et al., Proc. Natl. Acad. Sci. USA, 86:9079-9083, 1989.
Remington's Pharmaceutical Sciences, 18th Ed. Mack Printing Company, pp. 1289-1329, 1990.
Ridgeway, In: Vectors: A survey of molecular cloning vectors and their uses, Stoneham: Butterworth, pp. 467-492, 1988.
Ryman et al., J. Virol., 79(3):1487-99, 2005.
Sami et al., J. Infect. Dis., 171(2):440-3, 1995.
Samulski et al., EMBO J, 10:3941-3950, 1991.
Samulski et al., J Virol, 63:3822-3828, 1989.
Schultz-Chemy et al., Virology, 278(1):55-9, 2000.
Skiadopoulos et al., J. Virol., 78:6927-37, 2004.
Skiadopoulos et al., Virology, 345:492-501, 2006.
Szoka and Papahadjopoulos, Proc. Natl. Acad. Sci., 75:4194-4198, 1978.
Takimoto et al., J. Virol., 78(11):6043-7, 2004.
Tang et al., J. Virol., 77:10819-28, 2003.
Tang et al., Vaccine, 23:1657-67, 2005.
Tebbey et al., J. Exp. Med., 188(10):1967-72, 1998.
Temin, In: Gene Transfer, Kucherlapati (ed.), New York: Plenum Press, pp. 149-188, 1986.
Teng et al., Virology, 289(2):283-96, 2001.
Thompson & Sarnow, Curr. Opin. Microbio., 3:366 370, 2000.
Thompson et al. Proc. Natl. Acad. Sci. USA, 103:3722-7, 2006.
Thompson et al., Proc. Natl. Acad. Sci. USA, 103(10):3722-7, 2006.
Top et al., J. Infect. Dis., 124:155-160, 1971.
Tratschin et al., Mol. Cell. Biol., 5:3258-3260, 1985.
Tripp et al., Nat. Immunol., 2:732-8, 2001.
U.K. Patent Application GB 2193095 A
van den Hoogen et al., Nat. Med., 7:719-24, 2001
Varga et al., Immunity, 15:637-46, 2001.
Vaux-Peretz et al., Vaccine, 8(6):543-8, 1990.
Velders et al., Cancer Res., 61(21):7861-7, 2001.
Wagner et al., Science, 260:1510-1513, 1990.
Walsh, Vaccine, 11(11):1 135-8, 1993.
Walsh et al., J. Clin. Invest, 94:1440-1448, 1994.
Wang et al., Breast Cancer Res., 7(1):R145-55, 2005.
Waris et al., J. Virol., 70(5):2852-60, 1996.
Welliver et al., J. Infect. Dis., 170(2):425-8, 1994.
White et al., J. Virol., 81(19):10329-39, 2007.
White et al., J. Virol., 75(8):3706-18, 2001.
Williams et al. N. Engl. J. Med., 350:443-50, 2004.
Williams et al., J. Infect. Dis., 192:1061-5, 2005.
Williams et al., J. Infect. Dis., 192:1149-53, 2005.
Williamson et al., AIDS Res. Hum. Retroviruses, 19(2):133-44, 2003.
WO 98/07408
WO 99/18933
Wong et al., Gene., 10(2):87-94, 1980.
Wu and Wu, J. Biol. Chem., 262:4429-4432, 1987.
Wu and Wu, Adv. Drug Delivery Rev., 12:159-167, 1993.
Wyatt et al., Vaccine, 18(5-6):392-7, 1999.
Yim et al., Vaccine, 25:5034-40, 2007.
Yang et al., Proc. Natl. Acad. Sci. USA, 91:4407-4411, 1994.
Yoder et al., Bio/Technology 12:263-268, 1994.
You et al., Respir. Res., 7:107, 2006.
Zhou et al., J. Exp. Med., 179:1867-1875, 1994.

What is claimed is:

1. A paramyxovirus virus-like particle comprising paramyxovirus matrix protein (M), nucleoprotein (N), phosphoprotein (P) and fusion protein (F), but excluding all other paramyxovirus proteins.

2. The paramyxovirus virus-like particle of claim 1, wherein the paramyxovirus is respiratory syncytial virus, metapneumovirus, parainfluenza virus, or measles virus.

3. A method of producing a paramyxovirus virus-like particle comprising:
(a) providing one or more expression constructs encoding paramyxovirus matrix protein (M), nucleoprotein (N), phosphoprotein (P) and fusion protein (F);
(b) transferring said one or more expression constructs into a host cell; and
(c) culturing said host cell under conditions supporting expression of M, N, P and F proteins.

4. The method of claim 3, wherein each of said M, N, P and F are encoded on one distinct expression constructs, two distinct expression constructs or three distinct expression constructs.

5. The method of claim 3, wherein each of said M, N, P and F are encoded on four distinct expression constructs.

6. The method of claim 3, wherein said one or more expression constructs is a viral expression construct.

7. The method of claim 6, wherein said viral expression construct is an alphavirus construct.

8. The method of claim 3, wherein said host cell is a human cell.

9. The method of claim 3, wherein transferring comprises DNA gene gun transfer.

10. The method of claim 3, wherein said one or more expression constructs are comprised in a lipid formulation.

11. The method of claim 3, wherein the paramyxovirus is respiratory syncytial virus, metapneumovirus, parainfluenza virus, or measles virus.

12. A method of inducing a paramyxovirus immune response in a subject comprising:
(a) providing one or more expression constructs encoding paramyxovirus matrix protein (M), nucleoprotein (N), phosphoprotein (P) and fusion protein (F);
(b) administering said one or more expression constructs to said subject.

13. The method of claim 12, wherein each of said M, N, P and F are encoded on one distinct expression constructs, two distinct expression constructs or three distinct expression constructs.

14. The method of claim 12, wherein each of said M, N, P and F are encoded on four distinct expression constructs.

15. The method of claim 12, wherein said expression construct is a viral expression construct.

16. The method of claim 15, wherein said viral expression construct is an alphavirus construct.

17. The method of claim 12, wherein said subject is a human subject.

18. The method of claim 12, wherein transferring comprises DNA gene gun transfer.

19. The method of claim 12, wherein said one or more expression constructs are comprised in a lipid formulation.

20. The method of claim 12, wherein administering comprises intramuscular or intradermal injection.

21. The method of claim 17, wherein said human subject is an infant or is immunocompromised.

22. The method of claim 12, wherein the paramyxovirus is respiratory syncytial virus, metapneumovirus, parainfluenza virus, or measles virus.

23. The method of claim 12, further comprising assessing an immune response to M, N, P and/or F proteins in said subject.

24. A method of immunizing a subject comprising administering to said subject a paramyxovirus virus-like particle comprising matrix protein (M), nucleoprotein (N), phosphoprotein (P) and fusion protein (F), but excluding all other paramyxovirus proteins.

25. The method of claim 24, wherein the paramyxovirus is respiratory syncytial virus, metapneumovirus, parainfluenza virus, or measles virus.

26. The method of claim 24, wherein administering comprises intramuscular or intradermal injection.

27. The method of claim 24, wherein said subject is a human subject.

28. The method of claim 27, wherein said human subject is an infant or is immunocompromised.

29. The method of claim 24, further comprising administering to said subject an adjuvant.

30. The method of claim 24, further comprising assessing an immune response to M, N, P and/or F proteins in said subject.
